(19) United States
(12) Patent Application Publication
(10) Pub. No.: US 2022/0072271 A1
(43) Pub. Date: Mar. 10, 2022

(54) CATHETER

(71) Applicant: RESEARCH AND BUSINESS FOUNDATION SUNGKYUNKWAN UNIVERSITY, Suwon-si (KR)

(72) Inventors: Seongsu CHO, Hanam-si (KR); Ryungeun SONG, Gangneung-si (KR); Jinkee LEE, Suwon-si (KR)

(73) Assignee: RESEARCH AND BUSINESS FOUNDATION SUNGKYUNKWAN UNIVERSITY, Suwon-si (KR)

(21) Appl. No.: 17/527,692

(22) Filed: Nov. 16, 2021

Related U.S. Application Data
(63) Continuation of application No. PCT/KR2020/006024, filed on May 7, 2020.

(30) Foreign Application Priority Data
May 21, 2019 (KR) 10-2019-0059399

(51) Int. Cl. A61M 25/00

(52) U.S. Cl. CPC A61M 25/0029; A61M 2025/0037; A61M 2025/0031

(57) ABSTRACT

A catheter includes a first lumen and a second lumen in which fluid flows, and a longitudinal end part including a body portion having a constant diameter and a nozzle portion extending from a longitudinal end of the body portion with a gradually reducing diameter. A side surface of the body portion includes cavities fluidly communicating with the first lumen and the second lumen.

### CROSS-REFERENCE TO RELATED APPLICATIONS

Pursuant to 35 USC 120 and 365(c), this application is a continuation of International Application No. PCT/KR2020/006024 filed on May 7, 2020, which claims the benefit of Korean Patent Application No 10-2019-0059399 filed on May 21, 2019, in the Korean Intellectual Property Office, the entire disclosures of which are incorporated herein by reference for all purposes.

### BACKGROUND

### 1. Field

The following description relates to a catheter.

### 2. Description of Related Art

Central venous catheters (CVCs) are widely used for hemodialysis. Among the catheters, catheters having symmetrical tips are most widely used.

Hemodialysis using such a catheter is performed using a hemodialysis machine in a state in which the catheter is inserted into a patient's central vein, and progresses by a method of introducing blood into a first lumen through an inlet formed at a longitudinal end of the catheter, processing the blood introduced through the first lumen using the hemodialysis machine, and discharging the blood into a second lumen through an outlet formed at the longitudinal end of the catheter as well.

In this case, a recirculation phenomenon occurs in which the blood, which has been dialyzed and discharged through the outlet formed at the longitudinal end of the catheter, is reintroduced into the inlet, thereby reducing the efficiency of the hemodialysis.

Further, in the course of using the catheter, shear stress adversely affects blood cells, and thus increases the probability of formation of a thrombus. This clogs the inside of the catheter, resulting in loss of function of the catheter, and increases the cost for replacing the catheter, and increases the risk of cannulation when the catheter is replaced.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a catheter includes a first lumen and a second lumen in which fluid flows, and a longitudinal end part including a body portion having a constant diameter and a nozzle portion extending from a longitudinal end of the body portion with a gradually reducing diameter. A side surface of the body portion includes cavities fluidly communicating with the first lumen and the second lumen.

In a longitudinal end surface of the nozzle portion, an inlet may fluidly communicate with the first lumen through which the fluid is introduced, and an outlet may fluidly communicate with the second lumen through which the fluid is discharged. The catheter may further include a separation wall extending in an elongated manner from a partition wall partitioning the first lumen and the second lumen at the longitudinal end of the nozzle portion, and separates the inlet and the outlet.

An inlet fluidly communicating with the first lumen through which the fluid is introduced, and an outlet fluidly communicating with the second lumen through which the fluid is discharged may be formed in a shape in which a portion between a longitudinal end of the nozzle portion and a side surface of the nozzle portion may have a curved surface in a longitudinal direction.

An inclination of the curved surface may increase from a longitudinal end surface of the nozzle portion towards side surfaces of the nozzle portion.

An inlet fluidly communicating with the first lumen through which the fluid is introduced and an outlet fluidly communicating with the second lumen through which the fluid is discharged may be symmetrically formed to have a shape in which a portion between a longitudinal end surface of the nozzle portion and a side surface of the nozzle portion may have a diagonal cut.

A longitudinal end surface of the nozzle portion may include a partition wall having an I-beam shape partitioning the first lumen and the second lumen.

The longitudinal end surface of the nozzle portion may include a partition wall having an I-beam shape partitioning the first lumen and the second lumen.

Each of the cavities may have an oval shape.

A first one of the cavities may fluidly communicate with the first lumen, and another one of the cavities may fluidly communicate with the second lumen.

The first one of the cavities may be disposed opposite to the other one of the cavities.

Other features and aspects will be apparent from the following detailed description, the drawings, and the claims.

### BRIEF DESCRIPTION OF THE DRAWINGS

FIG. 1 is a perspective view showing a longitudinal end part of a catheter according to a first embodiment of the present disclosure.

FIG. 2 is a side view of FIG. 1.

FIGS. 3A and 3B show a particle tracing simulation result according to the presence or absence of a hole in a side surface of a body.

FIG. 4 shows a flow analysis result according to the shape of the hole formed on the side surface of the body.

FIGS. 5A and 5B show plan views of the longitudinal end part of the catheter when there is no nozzle portion or when there is a nozzle portion as in the present disclosure.

FIGS. 6A and 6B show a flow rate analysis result and a blood damage index (BDI) result according to the shape of the catheter of FIGS. 5A and 5B, respectively.

FIGS. 7A and 7B show a change in shear stress according to the shape of the catheter of FIGS. 5A and 5B, respectively.

FIG. 8 is a perspective view showing a longitudinal end part of a catheter according to a second embodiment of the present disclosure.

FIG. 9 is a side view of FIG. 8.

FIG. 10 is a plan view of FIG. 8.

FIG. 11 is a perspective view showing a longitudinal end part of a catheter according to a third embodiment of the present disclosure.

FIG. 12 is a side view of FIG. 11.

FIG. 13 is a plan view of FIG. 11.

FIG. 14 is a perspective view showing a longitudinal end part of a catheter according to a fourth embodiment of the present disclosure.

FIG. 15 is a side view of FIG. 14.

FIG. 16 is a plan view of FIG. 14.

FIG. 17 shows a particle tracing simulation result of the catheter according to the third embodiment.

FIG. 18 is a schematic view of the shape of a cut surface of the longitudinal end part of the catheter according to the third embodiment.

FIG. 19 shows simulation results according to the existing Palindrome and Glidepath models and models according to the second to fourth embodiments of the present disclosure and dye tracing experimental results.

FIGS. 20A and 20B are a graph of a recirculation rate and a BDI obtained based on the results in FIG. 19.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

Due to manufacturing techniques and/or tolerances, variations of the shapes shown in the drawings may occur. Thus, the examples described herein are not limited to the specific shapes shown in the drawings, but include changes in shape that occur during manufacturing.

The features of the examples described herein may be combined in various ways as will be apparent after an understanding of the disclosure of this application. Further, although the examples described herein have a variety of configurations, other configurations are possible as will be apparent after an understanding of the disclosure of this application.

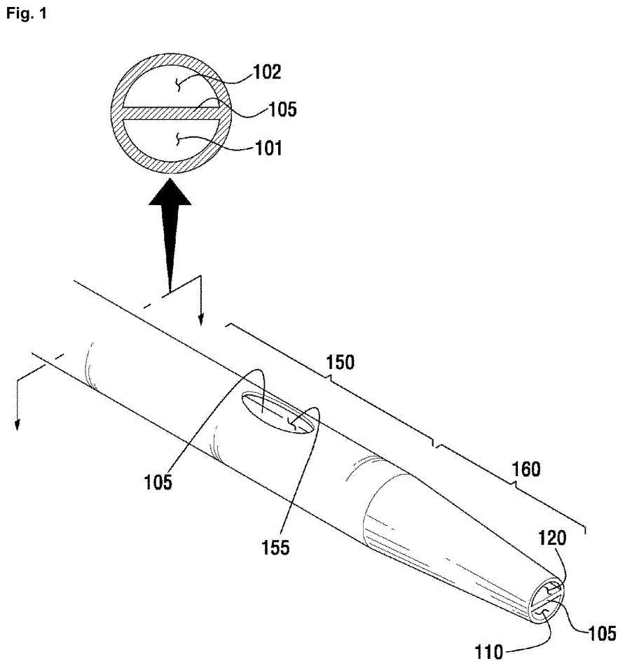

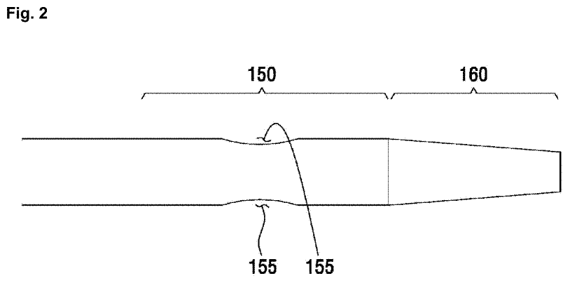

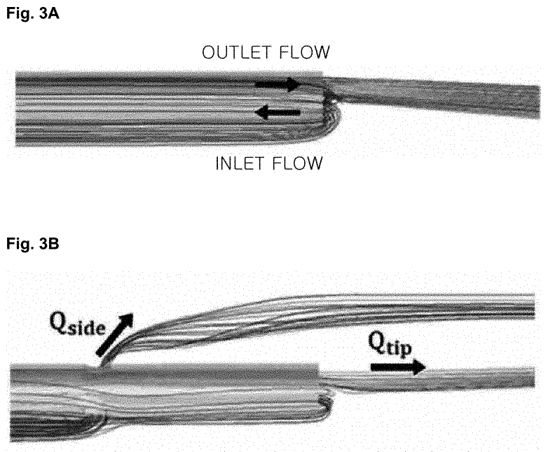

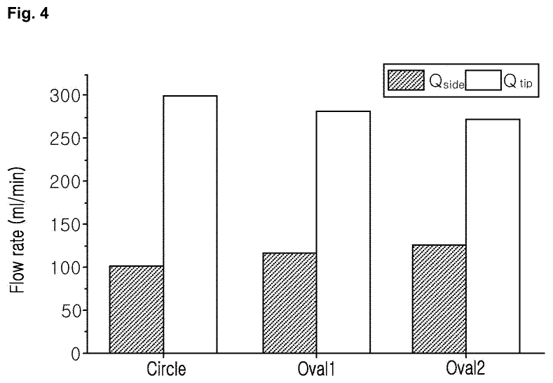

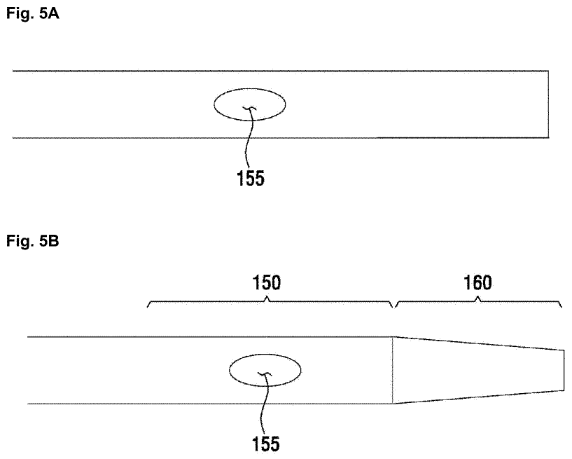

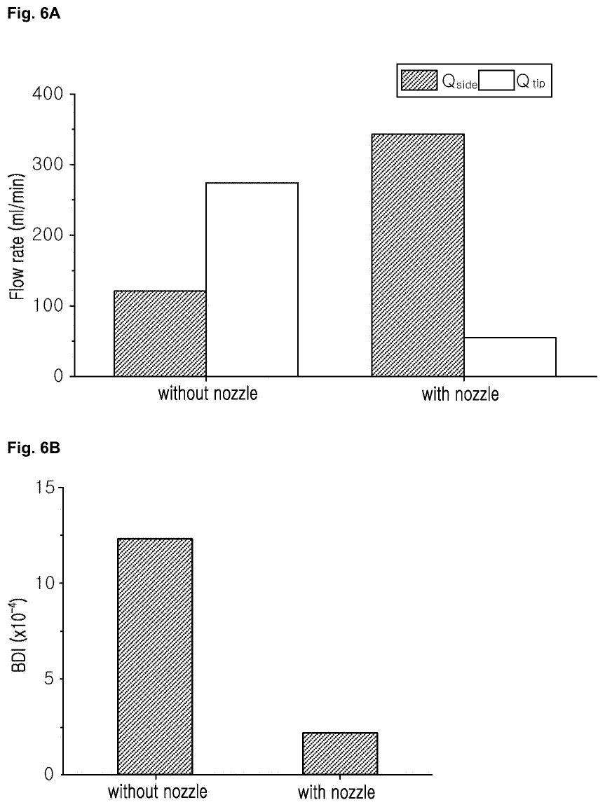

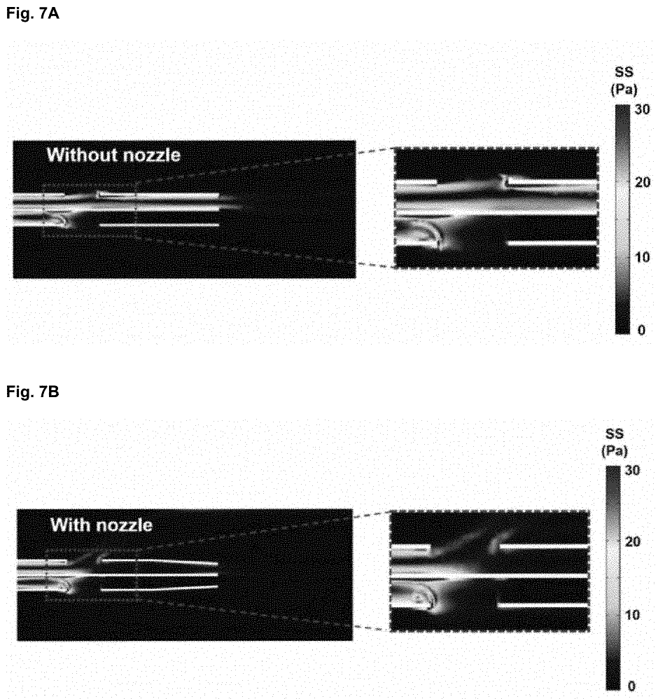

FIG. 1 is a perspective view showing a longitudinal end part of a catheter according to a first embodiment of the present disclosure. FIG. 2 is a side view of FIG. 1. FIGS. 3A and 3B show a particle tracing simulation result according to the presence or absence of a hole in a side surface of a body. FIG. 4 shows a flow analysis result according to the shape of the hole formed on the side surface of the body. FIGS. 5A and 5B show plan views of the longitudinal end part of the catheter when there is no nozzle portion or when there is a nozzle portion as in the present disclosure. FIGS. 6A and 6B show a flow rate analysis result and a blood damage index (BDI) result according to the shape of the catheter of FIGS. 5A and 5B. FIGS. 7A and 7B show a change in shear stress according to the shape of the catheter of FIGS. 5A and 5B.

According to the present disclosure, the catheter is formed with a first lumen 101 and a second lumen 102 therein through which a fluid flows. In this case, the first lumen 101 and the second lumen 102 may be partitioned by a partition wall 105 formed along the center of a circular tube. Thus, respective openings thereof may be formed to have a semicircular shape, but the present disclosure is not limited thereto.

In this case, as described below, an inlet 110 connected to the first lumen 101 and an outlet 120 connected to the second lumen 102 are formed in the longitudinal end part of the catheter. Thus, the blood introduced into the catheter through the inlet 110 flows through the first lumen 101 and is processed by a hemodialysis machine (not shown), and the processed blood flows through the second lumen 102 and is discharged through the outlet 120.

The longitudinal end part forming a tip of the catheter may include a body portion 150 and a nozzle portion 160, as shown in FIG. 1.

The body portion 150 has a constant diameter in the form of a circular tube. A cavity or hole 155 communicates with the first lumen 101. The second lumen is formed at a symmetrical point in the outer surface of the body portion 150. Thus, the blood may be introduced into the inside of the catheter through the hole 155 communicating with the first lumen 101. Further, the blood processed by the hemodialysis machine may be discharged outside through the hole 155 communicating with the second lumen 102 on an opposite side.

The nozzle portion 160 extends from a longitudinal end of the body portion 150 and is formed so that the diameter of a tube gradually decreases. In this case, the inlet 110 connected to the first lumen 101 and through which the blood is introduced and the outlet 120 connected to the second lumen 102 and through which the blood is discharged are formed in a longitudinal end surface of the nozzle portion 160.

Recirculation of the catheter may occur while flow discharged from the outlet 120 formed at the longitudinal end of the catheter and flow introduced into the catheter from the inlet 110 overlap each other. That is, when the inlet 110 and the outlet 120 are formed to be adjacent to each other, recirculation is likely to occur. However, as in the present disclosure, when the hole 155 is formed in the body portion 150, the blood discharged through the hole 155 and the outlet 120 connected to the second lumen 102 may be dispersed. Thus the flow rate of the blood discharged through the outlet 120 formed at the longitudinal end of the catheter may be reduced. Thus, the flow rate at the longitudinal end of the catheter may be reduced, and thus a probability that the recirculation occurs between the inlet 110 and the outlet 120 in the longitudinal end of the catheter may be reduced.

FIG. 3A shows a flow result using computer simulation when the hole 155 is not formed in the body portion 150, and FIG. 3B shows a flow result using computer simulation when the hole 155 is formed in the body portion 150 as described above. As shown in FIG. 3B, since the blood may be dispersed and discharged through the hole 155 formed in the body portion 150, it can be identified that a flow rate Qₜᵢₚ discharged through the outlet 120 formed at the longitudinal end of the catheter is much smaller than a simulation result of FIG. 3A in which there is no hole 155.

For reference, in the present disclosure, as will be described below, a recirculation rate and thrombus formation are analyzed using a particle tracing simulation during computer simulation. In the particle tracing simulation, the recirculation rate is calculated by counting the number of particles introduced into the catheter through the inlet 110 again among particles discharged from the outlet 120, and the thrombus formation is predicted by calculating a BDI value by analyzing shear stresses received by the respective particles. In this case, a pulsatile flow in consideration of a heart beat is applied so that the simulation may be performed to be similar to an actual blood flow, and the simulation is performed in which the size of the particles are also set to a size similar to that of red blood cells. In this case, the BDI is an index indicating the accumulation of damage to cells in the blood due to the shear stress, and as the BDI value becomes higher, the probability that more of the cells are damaged and thus a thrombus is formed becomes higher.

Further, the formation of the thrombus when the catheter is used is caused by the shear stress applied to the flow inside or outside the catheter, which occurs when the flow is concentrated in one place. Thus, as in the present disclosure, a flow rate Q_{side} discharged through the hole 155 of the body portion 150 and the flow rate Qₜᵢₚ discharged through the outlet 120 at the longitudinal end of the catheter may be dispersed, and thus the probability of formation of the thrombus may be reduced.

In this case, the hole 155 may be formed in the form of an oval forming a long axis in a longitudinal direction that is a lengthwise direction of the catheter.

FIG. 4 shows a simulation result of the flow rate Q_{side} discharged through the hole 155 of the body portion 150 and the flow rate Qₜᵢₚ discharged through the outlet 120 in a longitudinal end surface of the catheter when the hole 155 is formed in a circular shape (Circle in FIG. 4) and when the hole 155 is formed in an oval (Oval 1 and Oval 2 in FIG. 4). (For reference, Oval 2 is larger than Oval 1). It can be identified from FIG. 4 that when the hole 155 has an oval shape rather than a circular shape, the flow rate Q_{side} discharged through the hole 155 is large and the flow rate Qₜᵢₚ discharged through the outlet 120 at the end of the catheter is small. Thus, when the hole 155 is formed in an oval shape, the flow rate Qₜᵢₚ discharged through the outlet 120 at the end of the catheter may be reduced, and thus, as compared to a case in which the hole 155 is formed in a circular shape, the recirculation rate can be reduced, and the probability of formation of the thrombus may can be also reduced.

Next, in the present disclosure, the nozzle portion 160 that is tapered so that the diameter of the tube gradually decreases is formed at the longitudinal end of the catheter. FIG. 5A shows a case in which the diameter of the tube is constant without forming the nozzle portion 160 at the longitudinal end of the catheter as in the related art, and FIG. 5B shows a case in which the nozzle portion 160 is formed at the longitudinal end of the catheter as in the present disclosure. In FIGS. 5A and 5B, a simulation is performed for the catheter in which the hole 155 is formed in the side surface of the catheter, and FIGS. 6A and 6B show the simulation result therefor.

In FIG. 6A, it can be identified that when the nozzle portion 160 is formed as in the present disclosure, the flow rate Q_{side} discharged through the hole 155 of the body portion 150 is much larger and thus the flow rate Qₜᵢₚ discharged through the outlet 120 at the longitudinal end of the catheter is much smaller, as compared to a case in which the nozzle portion 160 is not formed. This identifies that the nozzle portion 160 serves as one kind of resistance in the flow of the blood, and thus the flow rate Q_{side} discharged through the hole 155 of the body portion 150 increases.

Further, in FIGS. 7A and 7B, as in the present disclosure, when the nozzle portion 160 is formed, the flow rate discharged through the hole 155 and the flow rate discharged through the outlet 120 may be properly dispersed, and thus it can be identified that a strong shear stress formed inside and around the catheter is reducible. Furthermore, in FIG. 6B, it can be identified that the shear stress is reduced, and thus the BDI value is also reduced. This means that the probability of formation of the thrombus is reduced.

In this way, as in the present disclosure, the nozzle portion 160, in which the diameter of the tube is gradually reduced, is formed at the longitudinal end part of the catheter, and the hole 155 is formed in the side surface of the body portion 150. Thus, the flow rate Qₜᵢₚ discharged from the outlet 120 at the longitudinal end of the catheter and the flow rate Q_{side} discharged through the hole 155 of the body portion 150 may be properly dispersed. Therefore, the recirculation rate and the probability of formation of the thrombus can be reduced. Preferably, as described above, the hole 155 may be formed in an oval shape that is long in the longitudinal direction of the catheter.

Further, when the flow between the outlet 120 and the inlet 110 formed in the longitudinal end surface of the catheter is separated as much as possible, the recirculation rate can be further improved, and the description thereof will be described below.

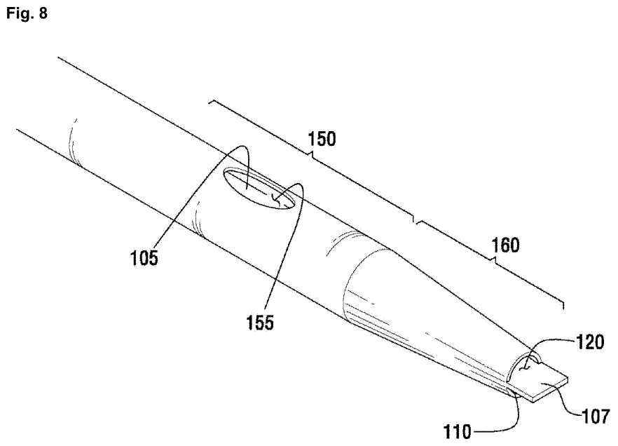

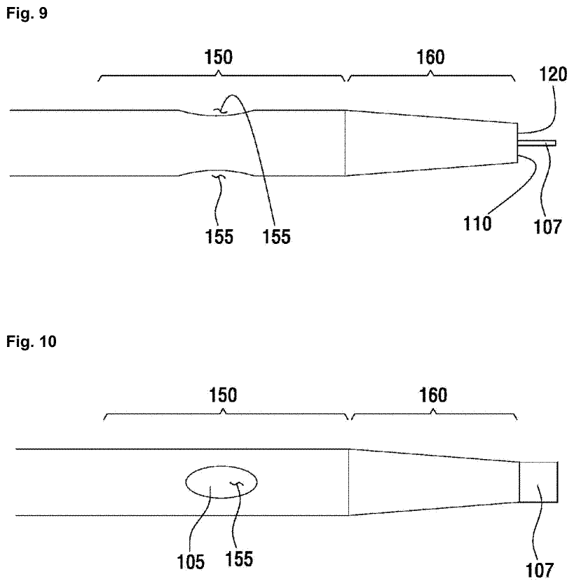

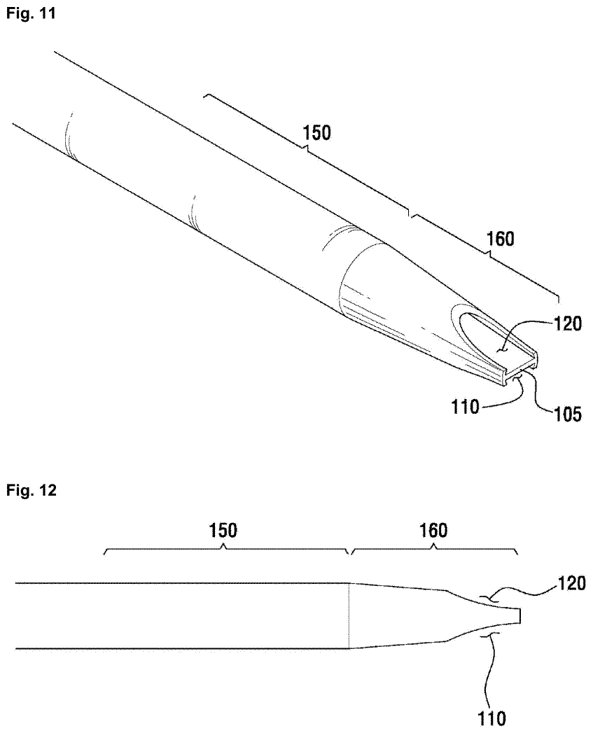

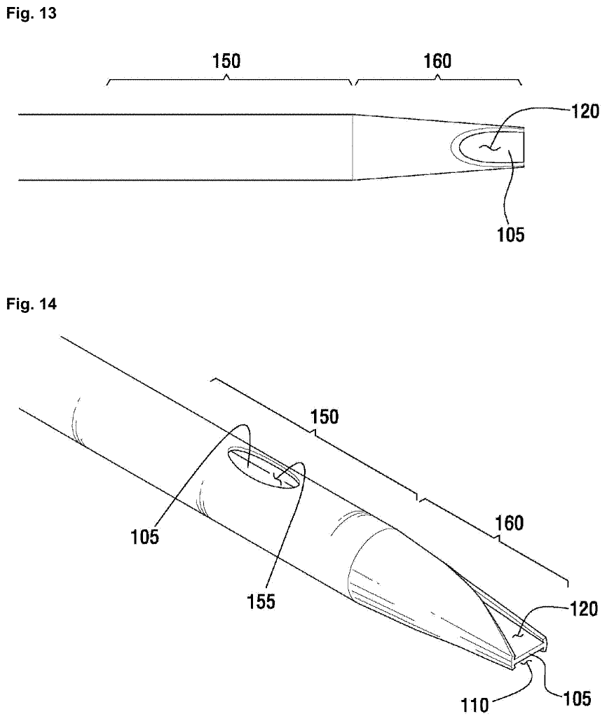

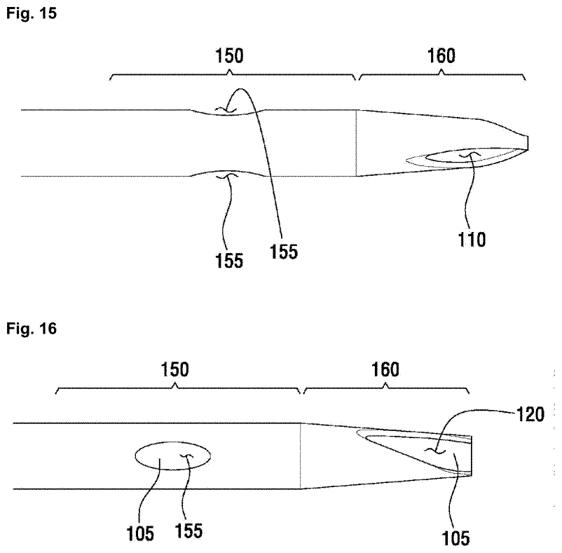

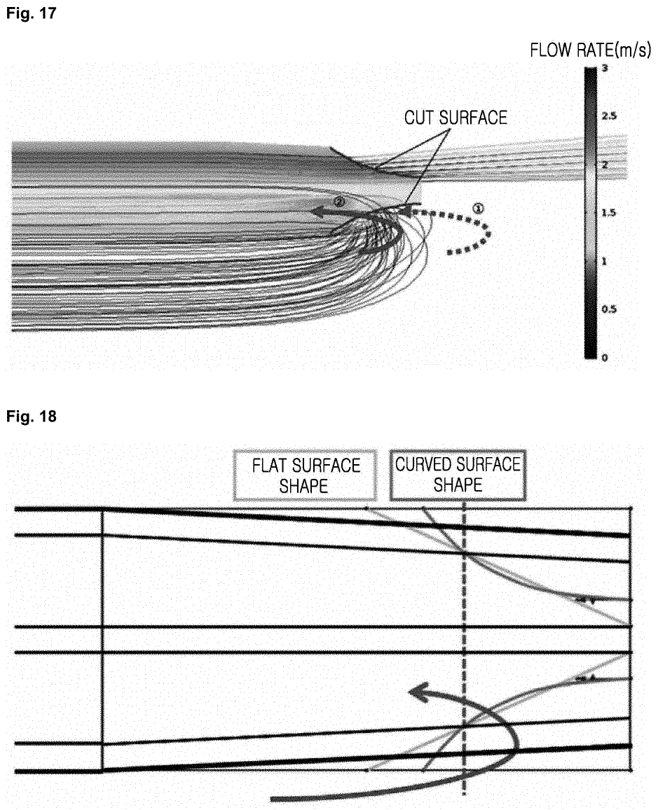

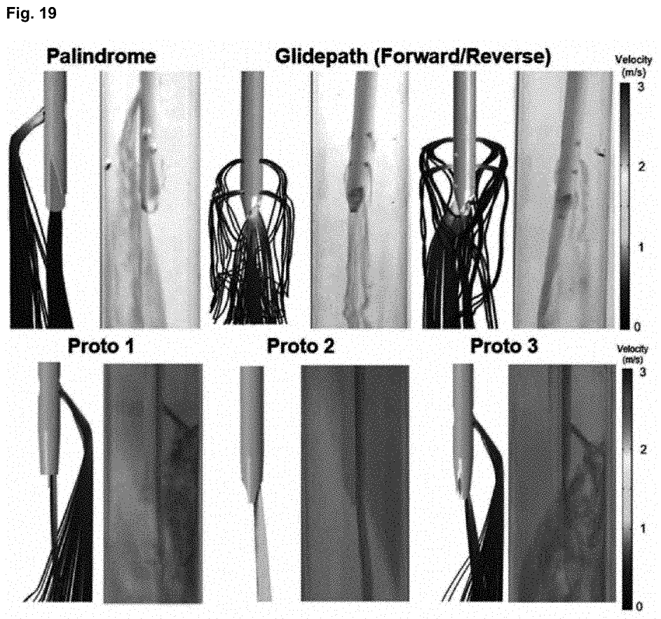

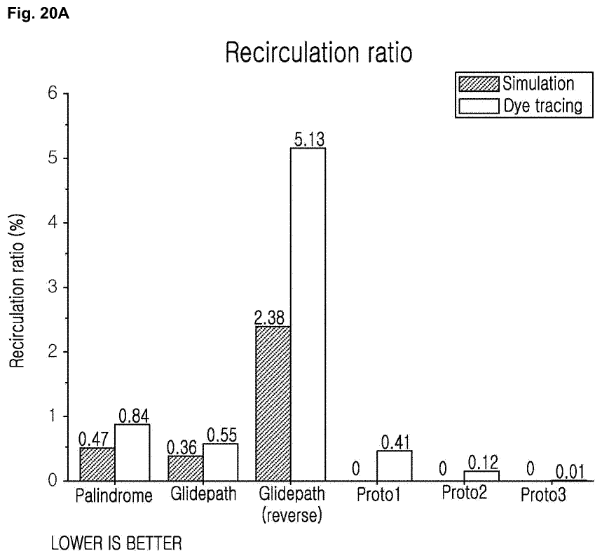

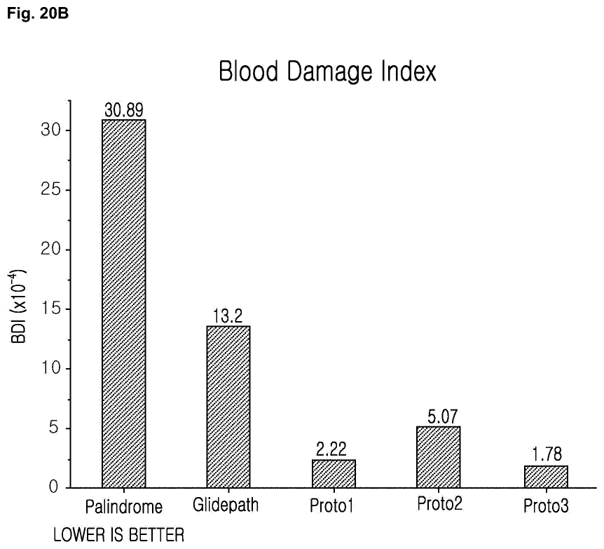

FIG. 8 is a perspective view showing a longitudinal end part of a catheter according to a second embodiment of the present disclosure, FIG. 9 is a side view of FIG. 8, FIG. 10 is a plan view of FIG. 8, FIG. 11 is a perspective view showing a longitudinal end part of a catheter according to a third embodiment of the present disclosure, FIG. 12 is a side view of FIG. 11, FIG. 13 is a plan view of FIG. 11, FIG. 14 is a perspective view showing a longitudinal end part of a catheter according to a fourth embodiment of the present disclosure, FIG. 15 is a side view of FIG. 14, FIG. 16 is a plan view of FIG. 14, FIG. 17 shows a particle tracing simulation result of the catheter according to the third embodiment, FIG. 18 is a schematic view of the shape of a cut surface of the longitudinal end part of the catheter according to the third embodiment, FIG. 19 shows simulation results according to the existing Palindrome and Glidepath models and models according to the second to fourth embodiments of the present disclosure and dye tracing experimental results, and FIGS. 20A and 20B are a graph of a recirculation rate and a BDI obtained on the basis of the results in FIG. 19.

According to the present disclosure, various embodiments related to the shape of the catheter will be described first with reference to FIGS. 8 to 16, and then simulations and experimental results thereof will be described with reference to FIGS. 17 to 20B.

As described with reference to FIGS. 1 to 7B, the features of the configuration in which the longitudinal end part of the catheter includes the body portion 150 and the nozzle portion 160 and in which the hole 155 is formed in the side surface of the body portion 150 are the same as those of the above-described embodiments. Thus, in the following description, differences from the above-described embodiments will be mainly described.

As shown in FIGS. 8 to 10, the catheter, according to the second embodiment of the present disclosure, has a separation wall 107 extending and protruding in an elongated manner from the partition wall 105, partitioning the first lumen 101 and the second lumen 102 on the longitudinal end surface of the nozzle portion 160. The separation wall 107 has a quadrangular shape, but the present disclosure is not limited thereto. The flow in which the blood is introduced from the inlet 110 and the flow in which the blood is discharged from the outlet 120 may be separated by the separation wall 107 as much as possible, thereby further reducing the recirculation rate.

Next, as shown in FIGS. 11 to 13, the catheter, according to the third embodiment of the present disclosure, has a shape in which portions between the longitudinal end of the nozzle portion 160 and middle portions of the side surfaces of the nozzle portion 160 are cut into a curved surface in the longitudinal direction that is the lengthwise direction of the catheter, and the outlet 120 and the inlet 110 are formed to be symmetrical to each other. In this case, as described above, the inlet 110 is connected to the first lumen 101 and the outlet 120 is connected to the second lumen 102. Thus, the partition wall 105 partitioning the first lumen 101 and the second lumen 102 is exposed to the outside, and the inlet 110 and the outlet 120 are also formed in a lateral direction of the catheter. Thus, when the blood flows through the inlet 110 and the outlet 120, the flow is separated by the partition wall 105 at an end of the catheter, thereby further reducing the recirculation rate. In this case, as shown in FIG. 12, the inlet 110 and the outlet 120 may be formed in a shape cut in a curved surface in the longitudinal direction. In more detail, as shown, the curved surface is formed so that the inclination thereof increases in a direction from the longitudinal end of the nozzle portion 160 toward the side surfaces of the nozzle portion 160. The curved surface will be described below.

Further, when the inlet 110 and the outlet 120 are formed in a shape cut between the longitudinal end of the nozzle portion 160 and the side surfaces of the nozzle portion 160 in the longitudinal direction, as shown in FIG. 11, the cut surfaces are formed at positions spaced upward and downward from the partition wall, and thus the longitudinal end surface of the nozzle portion 160 may be formed in an I-beam shape. That is, both edges of the cut tube and the partition wall 105 form an I shape. In this way, the longitudinal end surface of the nozzle portion 160 is formed in an I shape, thereby further improving the durability of the catheter.

For reference, FIGS. 11 to 13 show a case in which the hole 155 is not formed in the side surface of the body portion 150, and as described above, the oval hole 155 may be formed in the body portion 150.

Next, as shown in FIGS. 14 to 16, the catheter, according to the fourth embodiment of the present disclosure, is similar to the third embodiment described above in that the inlet 110 and the outlet 120 are formed in a shape in which the end of the nozzle portion 160 is cut. However, in the present embodiment, the inlet 110 and the outlet 120 are formed in a shape in which the longitudinal end of the nozzle portion 160 and the side surfaces of the nozzle portion 160 are cut in a diagonal direction rather than the longitudinal direction. As shown in FIG. 14, the inlet 110 and the outlet 120 are formed to be symmetrical to each other in a twisted doughnut shape in which, as the inlet 110 and the outlet 120 goes toward the longitudinal end, a width between open two sides forming the inlet 110 and the outlet 120 gradually increases, but the open positions are bent. Thus, even in the present embodiment, the partition wall 105 partitioning the first lumen 101 and the second lumen 102 is exposed to the outside, and the inlet 110 and the outlet 120 are formed to also be bent in the lateral direction of the catheter. Thus, when the blood flows through the inlet 110 and the outlet 120, the flow is separated by the partition wall 105 of the end, the blood is introduced and discharged while the flow is formed to be curved according to the shape of the inlet 110 and the outlet 120, and thus the recirculation rate can be further reduced.

Even in the present embodiment, as in the above-described embodiments, the longitudinal end surface of the nozzle portion 160 may be formed in an I shape.

FIG. 17 shows a simulation result in the third embodiment of the present disclosure. It can be identified that, when the inlet 110 and the outlet 120 are formed in the form in which a longitudinal end part of the nozzle portion 160 is cut, the position of the inlet 110 may be moved from \textcircled{1} to \textcircled{2}, and thus the flow introduced into the inlet 110 and the flow discharged from the outlet 120 may be separated more effectively, as compared to a case in which the inlet 110 and the outlet 120 are formed in the longitudinal end surface.

Further, as described above, the longitudinal end part of the nozzle portion 160 has a shape cut in the form of not a flat surface but a curved surface in the longitudinal direction. In general, since the shear stress increases in the cut surface, the wider the cut surface, the higher the probability of thrombus formation. Thus, as the cut surface is made deeper into the catheter, the recirculation rate can be reduced, but at the same time, the cut surface is also widened, thereby increasing the probability of formation of the thrombus. As shown in FIG. 18, when the cut surface is in the form of not a flat cut surface but a curved cut surface, the area of the cut surface may be reduced while achieving a similar recirculation prevention effect, and thus the probability of formation of a thrombus can be further reduced.

FIG. 19 shows simulation results between the conventional catheter models (Palindrome, Glidepath) and catheter models (In FIG. 19, Proto 1, Proto 2, and Proto 3 correspond to modeled catheters according to the second embodiment, the third embodiment, and the fourth embodiment) and dye tracing experimental results. In the dye tracing experiment, a dye-mixed solution is injected into the catheter, and thus the flow may be visualized, and the amount of the dye released into the outlet 120 and flowing back into the inlet 110 is measured, and thus the recirculation rate can be experimentally verified. For reference, in FIG. 19, the left side is the results according to the simulation and the right side is the results according to the dye tracing.

As can be seen in FIG. 19, it can be identified that there is no significant difference in the flow pattern when the results according to the simulation and the experimental results according to the dye tracing for each model are compared with each other. Further, the graph of FIGS. 20A and 20B show the results of obtaining the recirculation rate and the BDI value from FIGS. 19, and it can be identified that the recirculation rate and the BDI value are reduced in the case of the model according to the present disclosure as compared to the conventional model.

According to a catheter according to the present disclosure, due to features of an end tip of the catheter, a recirculation rate is lowered, and thus hemodialysis efficiency can be improved.

Further, by reducing the probability of formation of a thrombus, the lifetime of the catheter can be extended.

Further, since the lifetime of the catheter can be extended, costs of replacement can be reduced, and the risk of cannulation can be reduced during replacement.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A catheter, comprising:
a first lumen and a second lumen in which fluid flows; and
a longitudinal end part comprising a body portion having a constant diameter and a nozzle portion extending from a longitudinal end of the body portion with a gradually reducing diameter,
wherein a side surface of the body portion comprises cavities fluidly communicating with the first lumen and the second lumen.

2. The catheter of claim 1, wherein:
in a longitudinal end surface of the nozzle portion, an inlet fluidly communicates with the first lumen through which the fluid is introduced, and an outlet fluidly communicates with the second lumen through which the fluid is discharged; and
the catheter further comprises a separation wall extending in an elongated manner from a partition wall partitioning the first lumen and the second lumen at the longitudinal end of the nozzle portion and separates the inlet and the outlet.

3. The catheter of claim 1, wherein an inlet fluidly communicating with the first lumen through which the fluid is introduced, and an outlet fluidly communicating with the second lumen through which the fluid is discharged are formed in a shape in which a portion between a longitudinal end of the nozzle portion and a side surface of the nozzle portion has a curved surface in a longitudinal direction.

4. The catheter of claim 3, wherein an inclination of the curved surface increases from a longitudinal end surface of the nozzle portion towards side surfaces of the nozzle portion.

5. The catheter of claim 1, wherein an inlet fluidly communicating with the first lumen through which the fluid is introduced and an outlet fluidly communicating with the second lumen through which the fluid is discharged are symmetrically formed to have a shape in which a portion between a longitudinal end surface of the nozzle portion and a side surface of the nozzle portion has a diagonal cut.

6. The catheter of claim 3, wherein a longitudinal end surface of the nozzle portion includes a partition wall having an I-beam shape partitioning the first lumen and the second lumen.

7. The catheter of claim 5, wherein the longitudinal end surface of the nozzle portion includes a partition wall having an I-beam shape partitioning the first lumen and the second lumen.

8. The catheter of claim 1, wherein each of the cavities has an oval shape.

9. The catheter of claim 1, wherein a first one of the cavities fluidly communicates with the first lumen, and another one of the cavities fluidly communicates with the second lumen.

10. The catheter of claim 9, wherein the first one of the cavities is disposed opposite to the other one of the cavities.
